(19) United States
(12) Patent Application Publication
(10) Pub. No.: US 2003/0049283 A1
(43) Pub. Date: Mar. 13, 2003

(54) Scalp desensitizing formulation

(76) Inventor: Victor E. Vandell, Wilmington, DE (US)

Correspondence Address: PILLSBURY WINTHROP, LLP, P.O. BOX 10500, MCLEAN, VA, 22102, US

(21) Appl. No.: 09/942,672

(22) Filed: Aug. 31, 2001

(51) Int. Cl. A61K 31/24; A61K 35/78; A61K 7/06

(52) U.S. Cl. 424/401; 424/776; 514/536

(57) ABSTRACT

A method of desensitizing the scalp prior to a generally painful hair treatment or manipulation procedure by application of a composition containing a therapeutically effective amount of solubilized topical anesthetic and scalp/skin conditioning oils. The composition can be used for the conditioning and desensitization of scalp and skin areas which are irritated, agitated, or traumatized by hair treatment procedures like braiding, corn-rowing, electrolysis, hair plugging, hair removal or any other hair treatment procedure that causes pain to the hair shaft or follicle.

### BACKGROUND OF THE INVENTION

1. Field of the Invention

This invention relates to a method of desensitizing the scalp or other hairy body areas prior to undergoing a hair treatment or manipulation procedure and to a topical anesthetic composition useful in the desensitizing process.

2. Description of Related Art

Numerous hair treatment and manipulation procedures, such as, for example, bleaching, braiding, corn-rowing, dyeing, electrolysis, hair plugging, hair removal, hair relaxing or perming, waxing and other hair treatment procedures, can be irritating and painful to the individual receiving the treatment. Little or no attention has been directed to ways of effectively mitigating and reducing the not insubstantial pain resulting from these procedures.

A wide variety of anesthetic containing compositions has been described in the literature and is commercially available. None of these formularies are concerned with or are intended of use on the scalp to reduce or alleviate pain caused by hair treatments or manipulation procedures.

A water-based composition containing high concentrations of benzocaine in a solid micronized form which is dispersed or suspended in an emollient vehicle is described in U.S. Pat. No. 5,446,063 to Reuter et al. Selection of the emollient vehicle was limited to components which did not dissolve the benzocaine to any significant extent, apparently in order to avoid the problem of recrystallization of the benzocaine from solution.

Durbak et al. in U.S. Pat. No. 4,241,048 addressed the problem of recrystallization of benzocaine from powdered suspension products by utilizing a composition containing a linear copolymer of a vinylpyrrolidone and a long chain alpha-olefin.

Neither of the above discussed patents recognized the need for a topical anesthetic in a formulation which could be applied to the scalp prior to a painful hair treatment procedure.

### SUMMARY OF THE INVENTION

A wide variety of hair treatment and manipulation procedures can be painful or irritating to the recipient of that treatment or manipulation. Hair treatment procedures like braiding, corn-rowing, electrolysis, hair plugging, hair removal, waxing and other hair treatment procedures cause pain and/or irritation to the hair shaft or follicle.

The present invention is directed towards a method of reducing or alleviating the pain caused by the trauma inflicted by the hair treatment and manipulation procedures by applying a therapeutically effective amount of a topical anesthetic to the scalp prior to the pain inducing hair treatment.

The present invention is directed also towards an oil-based formulation which contains both a therapeutically effective amount of a solubilized topical anesthetic and scalp/skin conditioning oils. The formulation also contains a solubilizer for solubilizing the topical anesthetic, homopolymers, copolymers, and plant extract oils. This formulation can be used for the conditioning and desensitization of the scalp and/or skin area which is irritated, agitated, or traumatized by the hair treatment, manipulation or removal procedure.

The present invention is also directed to a method of desensitizing the scalp prior to a hair treatment by applying a topical anesthetic containing composition to the scalp, allowing the composition to remain on the scalp a long enough time to desensitize the treated area, and wherein the composition contains a therapeutically effective amount of the topical anesthetic.

Additionally, a method of desensitizing the scalp comprising applying a topical anesthetic containing composition to the scalp, permitting the composition to remain on the scalp thereby desensitizing the scalp, and then removing the composition from the scalp is provided.

The present invention provides a way to deliver a therapeutically effective amount of a topical anesthetic to the skin, especially the scalp, in an oil-based or lotion-based composition with minimal amounts of crystallization of the topical anesthetic from the composition.

### DETAILED DESCRIPTION OF THE INVENTION

The method of this invention for reducing or alleviating the pain generally associated with a wide variety of hair treatments or manipulation procedures includes applying a topical anesthetic composition to the scalp; allowing the topical anesthetic composition to remain on the scalp long enough for the topical anesthetic to desensitize the scalp; optionally, removing or rinsing away the topical anesthetic composition; and finally, starting the pain inducing hair treatment or manipulation. The topical anesthetic composition will preferably be an oil-based formulation with minimal amounts of crystallization of the topical anesthetic and will include various skin and scalp conditioning oils.

This composition provides the therapeutic effects of a topical anesthetic with scalp and skin conditioning oils which results in both the replenishment of scalp and skin oils and the desensitization of the scalp and skin epidermal and subcutaneous layers for pain relief during hair treatment procedures that irritate, agitate, or mildly traumatize the hair follicles, scalp and skin epidermal and subcutaneous layers. The present invention is suitable for application to the skin or scalp prior to procedures, such as hair braiding, corn-rowing, hair plugging, electrolysis, waxing and any other procedures that are reported to be painful due to the sensitivity of the scalp and other areas of the body covered with hair.

The topical anesthetic composition can also be applied via spraying as a mist onto large areas of the skin prior to treatment with hot wax to remove hair, for example, leg and bikini waxing procedures. The composition could also be applied onto the eyebrow prior to an eyebrow arching procedure. The anesthetic qualities of the composition should make both of these procedures less painful and more comfortable for the person undergoing the procedure.

It should be recognized that while the topical anesthetic formulation of the present invention is intended primarily for use on the scalp its use is not so limited. The formulation may be used on any area of the skin to which it is desired to deliver a topical anesthetic for alleviation of pain or irritation, specifically for pain alleviation prior to a hair treatment or manipulation process. The inclusion of scalp and skin conditioning components to the anesthetic formulation make it especially suited for use on the scalp.

The containment of the topical anesthetic, for instance, benzocaine, in an oil based solution is achieved by the use of a solubilizer component such as alkyl esters, for example, diethyl sebacate. Other possible solubilizers include the following alkyl esters, without limitation, diethyl adipate, diisopropyl adipate, dimethyl molonate, dibutyl sebacate, dibutyl adipate, diethyl pimelate, diethyl suberate, diethyl azelate, methyl ethyl succinate, diethyl isosuccinate, and other structurally similar solubilizers to the ones listed here can be used as well.

In order to effectively solubilize the topical anesthetic in the formulation, the solubilizer should be present in the formulation at a range of about 20 to about 50 weight percent based on the total weight of the formulation. Particularly preferred is use of the solubilizer at a concentration of about 30 to about 40 weight percent of the total formulation.

In contrast to Reuter et al.'s '063 patent, in the present invention, dissolution of the topical anesthetic in the composition is desired to be at a high enough level to be therapeutically effective while also avoiding the problem of recrystallization from solution.

The addition of low molecular weight polymers to the formulation also promotes the solubility of the topical anesthetic. Crystal growth suppression is obtained through the subsequent addition of homopolymers and copolymers.

Copolymers with molecular weights in the range of about 5000 to about 10,000 are particularly beneficial to the present formulation. Preferably, the copolymer is present in the formulation in a weight percent range of about 7 to about 10 weight percent of the total formulation. PVP-(polyvinylpyrrolidone)-1-hexadecene and PVP-eicosene copolymer, as disclosed in U.S. Pat. No. 4,241,048 are just some of the possible copolymers useful in the present invention. Homopolymers with average molecular weights (M_{w}) in the range of about 400 to about 3000 have been found to provide the needed solubility characteristics for the present formulation. Preferably, the homopolymer is present in the formulation in a weight percent range of about 20 to about 25 weight percent of the total formulation. Polyethylene glycol 400 monolauate, polyethylene glycol 1000 monoacachidate, polyethylene glycol 600 diarachidate, polyethylene glycol 1500 distearate, and polypropylene glycol, for instance, are just a few of the possible homopolymers that are useful as crystal growth suppression components, as set forth in U.S. Pat. No. 4,344,965

For benzocaine, the therapeutically effective amount needed to achieve ample desensitization of the scalp or skin is with the use of a composition having a benzocaine concentration of approximately 20 to 30 weight percent based on the total weight of the formulation. This amount has been found to be effective at relieving the pain associated with the hair treatment and manipulation procedures mentioned above.

The oil-based solubilizer allows for a compatible mixture of the topical anesthetic, such as benzocaine, with scalp and skin conditioning oils. A large variety of scalp/skin conditioning oils can be used such as ylang-ylang oil, tea tree oil, tangerine oil, mineral oil, sandalwood oil, cherry oil, jasmine oil, Egyptian musk oil, oriental musk oil, eucalyptus oil, peppermint oil and any other oil based plant extract commonly used for skin/scalp conditioning. A perfume oil, which may also be a plant extract oil, may also be incorporated into the formulation. Upon mixing of the solubilizer, anesthetic, homopolymers and copolymers, a slightly viscous oil develops. The addition of white petroleum, a thickening agent, increases the viscosity of the solution and at the same time also acts as a scalp and skin oil replenishing component. Other thickening agents that can be added to increase viscosity are hydrocarbon oils (e.g. mineral oil, liquid petrolatums), vegetable oils (e.g. coconut oil, linseed oil, cocoa butter oil), and fatty acid esters (e.g. isopropyl palmitate, butyl stearate). The concentrations of the various skin/scalp conditioning oils can be varied depending on desired conditioning properties.

The formulation is prepared by first mixing the topical anesthetic, solubilizer, homopolymers and copolymers together and heating the mixture at about 80° C. while stirring. The perfume oil, skin conditioning oils and water or a thickening agent, depending on the type of composition desired, can be added after dissolution of all of the above components being heated has occurred.

At about 20 weight percent, benzocaine stays in solution even after the formulation is cooled to room temperature. A very small amount of recrystallization may be observed after 24 hours.

At higher concentrations of benzocaine, between about 22 to about 30 weight percent, some precipitation is observed in the mixture several hours after the mixture has cooled to room temperature. This overall recrystallization problem is easily remedied by reheating the mixture either in a hot water bath or briefly in a microwave oven (<10 seconds). Total resolubility is achieved for the reheated mixture. In some cases, the crystal growth does not reoccur after the second heating. This is an improvement over the known art.

The following examples are merely illustrative of the present invention and are not intended to limit the scope and breadth of the present invention.

The ingredients utilized in the examples below are available from commercial suppliers such as Aldrich Chemicals (Milwaukee, Wis.), Spectrum Chemicals (Gardenia, Calif.), Penta Chemicals (Livingston, N.J.) and ISP Technologies (Wayne, N.J.). The components utilized were all analytical grade or higher in purity with no more than 2% wt. impurities. The reagents used for preparation of the oil-based scalp/skin conditioner and desensitizer can be purchased from those suppliers. The poly(ethylene glycol) monolaurate has the empirical formula of CH₃(CH₂)₁₀CO(OCH₂CH₂)ₙ—OH (wherein n is 8 to 15) with an average Mₙ about 400 (m.w.>20,000 amu) and a boiling point greater than 260 C. The diethyl sebacate has the empirical formula C₂H₅O₂C(CH₂)₈CO₂C₂H₅ with a density of 0.963 g/ml and a boiling point of 312° C. The ethyl-p-aminobenzoate (Benzocaine) has the empirical formula C₉H₁₁NO₂ with a formula weight of 165.20 amu.

The 2-pyrrolidinone, 1-ethenylhexadecyl homopolymer (PVP/1-Hexadecene) has the empirical formula (C₂₂H₄₁NO)ₙ with average molecular weight of 7300 amu. The PVP/1-Hexadecene is purchased exclusively from ISP Technologies Inc. under the trade name Ganex V-216.

The oil conditioners can be purchased from any manufacturer that deals in scalp and skin oils. These oils were used as the pure extract from their source with no dilution.

### EXAMPLE 1

### Preparation of an Oil-Based Scalp/Skin Conditioner and Desensitizer

The following components were dissolved by stirring while heating in a water bath at 80° C. All of the following protocols are actual experiments developed for the production of the formulation.

20% wt Formulation of Benzocaine

| | | |
|---|---|---|
| Diethyl Sebacate | 4 | grams |
| PVP-Hexadecene | 1 | gram |
| PEG-400 | 3 | grams |
| Benzocaine | 2.5 | grams |

After the above components were dissolved, the following components were added to the resulting solution while still heating.

| | | |
|---|---|---|
| White petroleum | 1 | gram |
| Ylang-Ylang oil | 0.5 | gram |
| Tea Tree oil | 0.5 | gram |
| Tangerine oil | 0.5 | gram |

The solution was then cooled to room temperature. No recrystallization was observed in the solution.

### EXAMPLE 2

### Preparation of Oil-Based Scalp/Skin Conditioner and Desensitizer

The following components were dissolved by stirring while heating in a water bath at 80° C.

22% wt Formulation of Benzocaine

| | | |
|---|---|---|
| Diethyl Sebacate | 4 | grams |
| PEG-400 | 3 | grams |
| Benzocaine | 2.5 | grams |

After the above components were dissolved, the following components were added to the resulting solution while still heating.

| | | |
|---|---|---|
| White petroleum | 1 | gram |
| Tea Tree oil | 0.5 | gram |
| Tangerine oil | 0.5 | gram |

The solution was then cooled to room temperature. No recrystallization was observed in the solution.

### EXAMPLE 3

### Preparation of Oil-Based Scalp/Skin Conditioner and Desensitizer

The following components were dissolved by stirring while heating in a water bath at 80° C.

26% wt Formulation of Benzocaine

| | | |
|---|---|---|
| Diethyl Sebacate | 4 | grams |
| PVP-Hexadecene | 1 | gram |
| PEG-400 | 3 | grams |
| Benzocaine | 3.5 | grams |

After the above components were dissolved, the following components were added to the resulting solution while still heating.

| | | |
|---|---|---|
| White petroleum | 1 | gram |
| Tea Tree oil | 0.5 | gram |
| Tangerine oil | 0.5 | gram |

The solution was then cooled to room temperature. Some recrystallization was observed in the solution about one hour after cooling.

### EXAMPLE 4

### Preparation of Lotion

Dissolve the following components by stirring while heating to 80° C.

20% wt Formulation of Benzocaine

| | | |
|---|---|---|
| Diethyl Sebacate | 5 | grams |
| PVP-Hexadecene | 1 | gram |
| PEG-400 | 3 | grams |
| Benzocaine | 2.5 | grams |

After the above components have dissolved during heating, add 1 gram of water to the resulting solution while still heating. The solution is then cooled to room temperature. The resulting lotion was white and creamy with minimal recrystallization.

### EXAMPLE 5

### Application to Scalp

The oil composition from Example 1 was used after washing, detangling and drying hair. Subject's hair was parted (e.g. row parting for corn-rowing) to expose scalp. The oil formulation was applied directly to the subject's scalp up and down both sides of the parted row. The composition was allowed to remain on the scalp (for <1 minute) (numbness may or may not be felt by the subject). Subject's hair was then braided down the treated row. Subject reported that the pain usually associated with braiding hair was greatly reduced to minimal or no pain at all associated with the process. The oil may remain on the scalp to aid as a conditioning treatment for the epidermal layer of the skin.

### EXAMPLE 6

### Application to Scalp

The lotion composition from Example 2 was used several hours after a hair braiding procedure by rubbing several eyedropper drops onto tender areas around the base of the scalp. The composition can remain on the scalp indefinitely and does not need to be rinsed off as the lotion wears off over time. Subject reported that the pain usually associated with post braiding tenderness was reduced to a very tolerable level.

What is claimed is:

1. A formulation for desensitizing skin and hair follicles comprising
a therapeutically effective amount of a topical anesthetic;
a solubilizer for solubilizing said topical anesthetic;
homopolymers;
copolymers; and
plant extract oils.

2. A formulation according to claim 1 further comprising a thickener.

3. A formulation according to claim 2, wherein said thickener is replaced by water.

4. A formulation according to claim 1, wherein said topical anesthetic is at least one member selected from the group consisting of benzocaine, lidocaine, and prolocaine.

5. A formulation according to claim 4, where said topical anesthetic is benzocaine.

6. A formulation according to claim 1 wherein said solubilizer is at least one member selected from the group consisting of diethyl sebacate, diethyl adipate, diisopropyl adipate, dimethyl molonate, dibutyl sebacate, dibutyl adipate, diethyl pimelate, diethyl suberate, diethyl azelate, methyl ethyl succinate, and diethyl isosuccinate.

7. A formulation according to claim 6 wherein said solubilizer is diethyl sebacate or diethyl adipate.

8. A formulation according to claim 1 wherein said homopolymer is either polyethylene glycol or polypropylene glycol.

9. A formulation according to claim 1 wherein said copolymer is polyvinylpyrrolidone-hexadecene.

10. A formulation according to claim 1 wherein said plant extract oils comprise one or more members of a group consisting of tea tree oil, yling-yling oil, tangerine oil, chamomile oil, jasmine oil, Egyptian musk oil, sandalwood oil, coconut oil, and peppermint oil.

11. A formulation according to claim 10 wherein said plant extract oils provide fragrant properties to said oil-based formulation.

12. A formulation according to claim 1 wherein said thickener is white petroleum.

13. A formulation according to claim 4 wherein said homopolymer has a molecular weight in the range of about 400 to about 1000.

14. A formulation according to claim 5 wherein said copolymer has a molecular weight in the range of about 5000 to about 10,000.

15. A formulation according to claim 5 wherein said copolymer is present in a range of about 7 to about 10 weight percent based on the weight of the total formulation.

16. A formulation according to claim 4 wherein said homopolymer is present in a range of about 20 to about 25 weight percent based on the weight of the total formulation.

17. A formulation according to claim 10 wherein said plant extract oils are present in a range of about 11 to about 15 weight percent based on the weight of the total formulation.

18. A formulation according to claim 3 wherein said solubilizer is present in a range of about 30 to about 40 weight percent based on the weight of the total formulation.

19. A formulation according to claim 1 wherein said emulsion is a lotion.

20. A method of desensitizing the scalp prior to a hair treatment
applying a topical anesthetic containing composition to the scalp;
allowing the composition to remain on the scalp a long enough time to desensitize the treated area; and
wherein the topical anesthetic containing composition contains a therapeutically effective amount of a topical anesthetic.

21. A method of desensitizing the skin comprising applying the composition of claim 1 to the skin.

22. A method of desensitizing the scalp comprising
applying a topical anesthetic containing composition to the scalp;
permitting said composition to remain on the scalp thereby desensitizing the scalp;
removing said composition from the scalp.

23. A method according to claim 22, wherein said topical anesthetic containing composition comprises a therapeutically effective amount of a topical anesthetic.

24. A method according to claim 22, wherein a hair treatment procedure is performed after the composition is removed from the scalp.
